(19) United States
(12) Patent Application Publication
(10) Pub. No.: US 2005/0009204 A1
(43) Pub. Date: Jan. 13, 2005

(54) Multiplexed binding assays for receptor arrays

(76) Inventors: Ye Fang, Painted Post, NY (US); Yulong Hong, Painted Post, NY (US); Joydeep Lahiri, Painted Post, NY (US); Fang Lai, Painted Post, NY (US); Jinlin Peng, Painted Post, NY (US); Brian L. Webb, Painted Post, NY (US); Ann M. Ferrie, Painted Post, NY (US)

Correspondence Address: CORNING INCORPORATED, SP-TI-3-1, CORNING, NY, 14831, US

(21) Appl. No.: 10/639,718

(22) Filed: Aug. 12, 2003

Related U.S. Application Data
(60) Provisional application No. 60/486,592, filed on Jul. 11, 2003.

(51) Int. Cl. G01N 33/543

(52) U.S. Cl. 436/518

(57) ABSTRACT

A thematic microarray and methods for multiplexed binding assays using a cocktail solution of labeled ligands in the presence or absence of a target compound is provided. The methods enables researchers to screening compounds against multiple targets using a microarray format.

### CLAIM OF PRIORITY

The present Application claims benefit of priority from U.S. Provisional Application No. 60/486,592, filed on Jul. 11, 2003, the content of which is incorporated herein.

### RELATED APPLICATIONS

The present Application is related to co-assigned U.S. patent application Ser. No. 09/974,415, filed Oct. 9, 2001, and Ser. No. 09/854,786, filed May 14, 2001.

### FIELD OF THE INVENTION

The present invention relates to biological, biochemical or chemical binding assays performed on a solid surface. In particular, the invention pertains to the use of G-protein-coupled receptor (GPCR) microarrays for multiplexed screening and profiling of biological species, biochemical or chemical compounds.

### BACKGROUND

G-protein coupled receptors (GPCRs) represent the single most important class of drug targets—approximately 50% of current drugs target GPCRs; about 20% of the top 50 best selling drugs target GPCRs; more than $23.5 billion in pharmaceutical sales annually are ascribed to medications that address this target class. (Drews, J., “Drug Discovery: A Historical Perspective” Science 2000, 287, 1960-1963; Ma, P., and Zemmel, R., “Value of Novelty” Nat. Rev. Drug Discov. 2002, v. 1, 571-572.) GPCRs are associated with almost every major therapeutic category or disease class, including pain, asthma, inflammation, obesity, cancer, as well as cardiovascular, metabolic, gastrointestinal and central nervous system diseases. The tremendous significance of drugs targeting GPCRs lies in the physiological roles of GPCRs—as cell-surface receptors responsible for transducing exogenous signals into intracellular response(s). (Haga, T., and Berstein, G., eds., G-Protein-Coupled Receptors, CRC Press, Boca Raton, Fla., 1999.) In the human genome there are about 400-700 GPCRs; ligands for about 200 GPCRs have been discovered. (Pierce, K. L. et al., “Seven-Transmembrane Receptors.” Nat. Rev. Mol. Cell Biol. 2002, v. 3, 639-650.) Although there is very little conservation at the amino acid level among GPCR sequences, all the GPCRs share a characteristic motif consisting of seven distinct hydrophobic transmembrane regions (each 20-30 amino acids in length), an extracellular N-terminus, and an intracellular C-terminus.

The success of GPCRs as drug targets stems from the fact that the binding of natural ligands to their paired GPCR(s) can be moderated using appropriate small molecule drugs. (Ma, P., and Zemmel, R., “Value of Novelty,” Nat. Rev. Drug Discov. 2002, v. 1, 571-572.) Effective engineering of these drugs is, however, critical as aberrant binding to such a physiologically significant target class can lead to serious side effects. Structural data on GPCRs is limited and rational drug design is a significant challenge. Designing drugs that do not bind to non-targeted GPCRs is almost impossible. Currently, selectivity studies are conducted downstream in the drug discovery process—discarding compounds because of adverse binding at this stage makes the drug discovery process both expensive and time consuming. Given these considerations, and the strong possibility that so-called “orphan” GPCRs, recently discovered as a result of the sequencing of the human genome, may be valuable targets (Howard, A. D., et al., “Orphan G-Protein-Coupled Receptors and Natural Ligand Discovery.” TiPS 2001, v. 22, 132-140), there is a strong need for technologies that enable screening against multiple GPCRs simultaneously.

Given the importance of G-protein-coupled receptors as drug targets, a wide range of technologies has been developed to screen compounds against GPCRs. (e.g., Hemmila, I. A., and Hurskainen, P., “Novel Detection Strategies for Drug Discovery,” Drug Discov. Today 2002, 7, S152-S156.) The increased pace of target identification (Venter, J. C., et al. “The Sequences of the Human Genome,” Science 2001, v. 291, 1304-1351; Hopkins, A. L., and Groom, C. R., “The Druggable Genome,” Nat. Rev. Drug Discov. 2002, v. 1, 727-730) and the increasing size of compound libraries continues to drive the development of novel GPCR screening technologies (Schreiber, S. L., “Target-Oriented and Diversity-Oriented Organic Synthesis in Drug Discovery,” Science 2000, v. 287, 1964-1968). These assays can be classified into cell based and GPCR-membrane based assays. Despite the interest and the overwhelming number of current and future GPCR targets, few methods have been described for simultaneously studying multiple GPCRs. Recently, two groups of researchers have suggested that arrays of transiently transfected cell clusters or GPCR transfected cells on barcoded substrates could be used for multiplexed compound screening. (Ziauddin, J., and Sabatini, D. M., “Microarrays of Cells Expressing Defined cDNAs,” Nature 2001, v. 411, 107-110; Beske, O. E., and Goldbard, S., “High-throughput Cell Analysis Using Multiplexed Array Technologies,” Drug Discov. Today 2002, v. 7, s131-s135.)

The value of parallel analysis afforded by DNA microarrays (e.g., Schena, M., et al. “Quantitative Monitoring of Gene Expression Patterns with a Complementary DNA Microarray,” Science 1995, v. 270, 467-470) has inspired the development of protein arrays (e.g., Mitchell, P., “A Perspective on Protein Microarrays,” Nat. Biotechnol. 2002, v. 20, 225-229). Beyond the use of protein abundance profiling as an analogue to gene expression profiling, protein arrays offer the possibility of highly parallel investigations of protein-small molecule and protein-protein interactions. (MacBeath, G., and Schreiber, S. L., “Printing Proteins as Microarrays for High-throughput Function Determination,” Science 2000, v. 289, 1760-1763; Schweitzer, B., et al. “Immunoassays with Rolling Circle DNA Amplification: A Versatile Platform for Ultrasensitive Antigen Detection,” Proc. Natl. Acad. Sci. USA 2000, v. 97, 10113-10119; Fang, Y., et al. “Membrane Protein Microarrays,” J. Am. Chem. Soc. 2002, v. 124, 2394-2395; and Fang, Y., et al. “G Protein-Coupled Receptor Microarrays,” Chem. Biochem. 2002, v. 3, 987-991.)

While the importance of combinatorial approaches to drug design has been realized, however, the biological equivalent of combinatorial chemistry—multi-target screening using protein microarrays—has not. Multi-target screening maximizes the potential for effective matching of biological target space to chemical ligand space. Although, protein microarrays are naturally suited for testing compounds against multiple proteins simultaneously, some of the fundamental aspects of multiplexed bioassays using protein chips are yet to be demonstrated. One such fundamental aspect is the need for cocktails of labeled ligands for multiplexed competitive binding assays. Problems due to non-specific binding, cross reactivity, and the lack of general guidelines for choosing labeled ligands have deterred scientists from testing the feasibility of using mixtures of labeled ligands. As such, protein arrays may contain redundant elements—those against which a labeled ligand is not present in the cocktail. This limits the practical multiplexing capability of protein microarrays. Nowhere is the development of this general methodology more important and pertinent than for GPCRs. Hence a need exists for a new process that can offer a multiplexed binding assay format employing GPCR microarrays.

### SUMMARY OF THE INVENTION

The present invention describes the use of receptor microarrays for multiplexed compound profiling or screening involving the use of cocktails of self-assembled labeled ligands. Multi-GPCR target screening based on binding assays requires the use of self-assembled ligand cocktails. The invention also discloses several multiplexed assay formats, including: saturation assays for parallel K_{d} determination, competitive binding assays for parallel IC₅₀ determination (e.g., selective potency) of a single compound against multiple GPCR targets, or parallel IC₅₀ determination (e.g., relative potency) of multiple compounds against their corresponding GPCR targets, and competitive binding assays and displacement assays for compound screening. Compound selectivity screening and profiling using multiplexed binding assays is one of the main applications of GPCR microarrays.

According an aspect of the present invention, a thematic microarray of GPCRs is made for multiplexed compound screening and profiling. The microarray comprises: a plurality of GPCRs arranged on a substrate at positionally defined locations. In first embodiment, all known and orphan GPCRs (in human genome) are included in a single array. In a second embodiment, a representative index array may include a GPCR member selected from each subfamily of GPCRs. In a third embodiment, a selectivity panel array may include at least a single GPCR member selected from several related subfamilies of GPCRs. The selected GPCR members are either related to a certain physiological or pharmacological function, or specific tissue systems. In a fourth embodiment, a muta-genesis array may be composed of a GPCR and its variants or mutants, or its corresponding GPCRs originated from different species.

The present method and assay format utilizes saturation capabilities of the labeled ligands to bind with their paired receptors for profiling and screening in indirect binding assays which involve competitive binding of a test compound against labeled-ligands.

Multi-target binding assays according to the present invention employ cocktails containing labeled ligands capable of self-assembly with their respective receptors. The invention includes a cocktail solution of at least one labeled-ligand. The labeled-ligands in the cocktail solution are selected based on their individual binding ability to each GPCR microspot element in the microarray. Each labeled-ligand should bind to at least one GPCR element with a desired binding affinity (e.g., ˜0.1 nM to ˜20 nM) and specificity of at least 50% to 60%, and minimal cross-activity (e.g., ≦10%) with other GPCR elements in the array.

Since, multiplexed binding assays are technically challenging for several reasons, this invention addresses potential solutions to problems associated with labeled ligand binding specificity and affinity, assay buffer compatibility, content suitability, detection and data analysis. It is believed that the data described herein demonstrates for the first time the use of multiplexed binding assays on protein arrays of any kind.

In certain embodiments, each microspot in a microarray contains only one kind of probe-receptor. In other embodiments, each microspot contains a predominant kind of probe-receptor within a biological membrane, (e.g., a GPCR-membrane preparation obtained from a cell line over expressing the receptor). In an alternative embodiment, at least one microspot can contain at least two kinds of detectable probe-receptors. Preferably a method to detect the different kinds of receptors involves using simultaneously different labeled ligands with different tags; the tag can be physically or chemically distinguishable from each other. Each labeled ligand binds specifically with one of the detectable receptors.

Also disclosed is a GPCR array device having a variety of GPCR species associated with the support substrate; such receptor species may include, for instance, neurotensin receptor subtype 1, motilin receptor, delta2 opioid receptor, opioid-like receptor subtype 1, acetylcholine receptor subtype-1 (M1), and control cell membranes of CHO or HEK cell lines.

Additional features and advantageous of the present invention will be revealed in the following detailed description. Both the foregoing summary and the following detailed description and examples are merely representative of the invention, and are intended to provide an overview for understanding the invention as claimed.

### BRIEF DESCRIPTION OF THE FIGURES

FIG. 1 is a schematic representation of an embodiment of the present invention, in which a multiplexed competitive binding assay uses a cocktail solution of labeled ligands with a GPCR micro array.

FIGS. 2A-C are schematic representations of a GPCR microarray in a 96-well microplate.

FIGS. 3A-C are concentration dependent total and nonspecific binding graphs showing a multiplexed saturation assay and K_{d} determination of fluorescent ligands using GPCR microarrays. The GPCR microrrays have three receptors: human neurotensin receptor subtype 1 (NTR1), δ2 opioid receptor (OP1) and motilin receptor (MOTR). A cocktail solution of three fluorescently-labeled ligands includes: Cy5-neurotensin 2-13 (Cy5-NT) to NTR1, Cy5-naltrexone to OP1, and Bodipy-TMR-motilin 1-16 (BT-MOT) to MOTR. The K_{d} values for these three ligands binding with their corresponding receptors in the microarrays were found to be about 2.5 nM for Cy5-NT binding to NTR1, about 1.9 nM for Cy5-naltrexone binding to OP1, and about 3 nM for BT-MOT to MOTR. These K_{d} values were essentially same as those obtained independently using single GPCR microarrays or other homogeneous assay technologies.

FIGS. 4A-C are concentration dependent inhibition graphs showing a selective potency determination of naltrexone to delta2 (OP1) (A), MOTR (B) and NTR1 (C) in the microarrays against a cocktail labeled ligands (Cy5-NT, BT-MOT and Cy5-naltrexone). The GPCR microrrays have three receptors, human neurotensin receptor subtype 1 (NTR1), delta2 opioid receptor (OP1) and motilin receptor (MOTR). A cocktail solution of ligands includes three fluorescently-labeled ligands: Cy5-NT to NTR1, Cy5-naltrexone to OP1, and BT-MOT to MOTR. The selective potency of naltrexone to these three receptors is consistent with what reported in literature. Naltrexone is known antagonist to delta2 only.

FIGS. 5A-C are concentration dependent inhibition graphs showing a multiplexed potency determination of cocktail compounds to GPCR microarrays using competitive binding assays and a cocktail labeled ligand, according to the present invention. In this case, several compounds, each binding specifically only to its paired receptor(s), are mixed together and used for relative potency studies. The GPCR microrrays have three receptors, human neurotensin receptor subtype 1 (NTR1), delta2 opioid receptor (OP1) and motilin receptor (MOTR). A cocktail solution of ligands includes three fluorescently-labeled ligands: Cy5-NT to NTR1, Cy5-naltrexone to OP1, and BT-MOT to MOTR. The IC₅₀ values obtained are similar to those reported in the scientific literature.

FIGS. 6A and 6B are graphs presenting data for a multiplexed compound screening using multiplexed competitive binding assays, according to the present invention. FIG. 6A shows histograms for selective inhibition of binding of Bodipy-TMR-motilin 1-16 (BT-MOT) and Cy5-neurotensin 2-13 (Cy5-NT) in a cocktail solution to a microarray having NTR1 and MOT receptors. BT-MOT and Cy5-NT are ligands known to bind to the MOTR and NTR1 receptors, respectively. When excess unlabeled neurotensin (NT) (1 μM) is used, binding to the NTR1 receptor microspot is suppressed. In the presence of excess MOT (1 μM), binding to the MOTR microspots is suppressed. When both unlabeled ligands were present, suppression of binding to both the GPCRs is observed. Binding of BT-MOT and Cy5-MOT is observed using a GenePix 4000A scanner (Axon Instruments) in the Cy3 and Cy5 channels, respectively. FIG. 6B shows the selective inhibition of the binding of BT-CGP 12177 and Cy5-naltrexone to a microarray of P 1 and 62 receptors. BT-CGP can only specifically bind to β1 in the arrays; Cy5-naltrexone can only specifically bind to 62 receptor. Naltrexone is an antagonist to 62, while CGP 12177 is a partial agonist to beta1 receptor. Signals of two channels (Cy3 and Cy5) are examined as a function of different compounds (CGP 12177, naltrexone, and both) at 1 μM.

FIGS. 7A and 7B present data for multiplexed compound screening using single labeled-ligand competitive binding assays according to the present invention. FIG. 5A depicts two false-color fluorescence images for selective binding and inhibition to arrays of an adrenergic receptor family. FIG. 5A(i) is a fluorescence image of a microarray having three columns consisting of β1, β2 and α2A adrenergic receptors treated with a solution containing BT-CGP (5 nM). FIG. 5A(ii) is an image of the array after treatment with a solution containing BT-CGP (5 nM) and ICI118551 (10 nM). FIG. 5B is a graph showing histogram analysis of the binding (BT-CGP) and inhibition (in the presence of ICI118551).

FIGS. 8A-D show a series of false-color images according to a method by which a displacement assay for compound screening is performed. Multiple arrays of β1 adrenergic receptor, NTR1, and dopamine D1 receptors are used. FIGS. 8A and 8B, respectively are fluorescence images of a microarray before and after incubation with BT-NT (2 nM). FIGS. 8C and 8D are fluorescence images showing the displacement of pre-bound BT-NT by unlabeled compounds. The microarray is first incubated with a solution of BT-NT (2 nM), washed, dried, and then imaged. This is followed by incubation with a second solution containing either CGP 12177 (10 μM) or NT (10 μM).

FIG. 9 shows a series of false-color fluorescence images of three columns of microspots in a microarray after the binding of Cy5-neurotensin 2-13 and BT-motilin 1-16 at different concentrations (16/16, 8/8, 4/4, 2/2, 1/1, 0.5/0.5 nM) in the absence (top) and presence (bottom) of 2 μM neurotensin and 2 μM motilin. Each column has four replicate microspots. From left to right, the column contains a) MOTR, b) NTR1, and c) a mixture of NTR1 and MOTR.

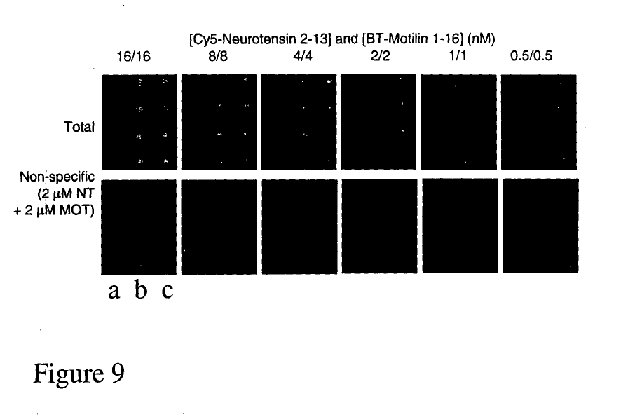

FIGS. 10A-D show saturation binding curves of a Cy5-neurotensin 2-13 and BT-Motilin 1-16 cocktail to MOTR and NTR1/MOTR on microarrays in the Cy-3 and Cy-5 detection channels. FIG. 10A shows the relative binding affinity of BT-Motilin 1-16 to microspots containing MOTR in either the absence (total signal) or presence (non-specific) of a mixture of neurotensin and motilin. FIG. 10B is a Scatchard plot of the data shown in FIG. 10A. FIG. 10C shows the relative binding affinity of BT-Motilin 1-16 to microspots containing the mixture of MOTR and NTR1 in either the absence (total signal) or presence (non-specific) of a mixture of neurotensin and motilin. FIG. 10D is a Scatchard plot of the data shown in FIG. 10C. The K_{d} value obtained for BT-Motilin 1-16 with MOTR and the mixture of MOTR and NTR1 are similar; however, the total binding sites are almost two-fold higher for MOTR alone than those for the MOTRINTR1 mixture.

### DETAILED DESCRIPTION OF THE INVENTION

### Section I—Definitions

Before describing the present invention in detail, this invention is not necessarily limited to specific compositions, reagents, process steps, or equipment, as such may vary. As used in this specification and the appended claims, the singular forms “a,” “an,” and “the” include plural referents unless the context clearly dictates otherwise. It is also to be understood that the terminology used herein is for the purpose of describing particular embodiments only, and is not intended to be limiting. All technical and scientific terms used herein have the usual meaning conventionally understood by persons skilled in the art to which this invention pertains, unless context defines otherwise.

The term “ligand” refers to a chemical molecule or biological molecule that can bind readily to a receptor with a specific binding affinity constant.

The term “labeled-ligand” refers to either a fluorescently labeled or radioactive isotope-labeled or hapten-labeled (e.g., biotin) or gold-nano-particle labeled ligand.

The term “cocktail solution” refers to a medium (e.g., buffered or aqueous solution) having a mixture either of different labeled ligands or of different compounds. Alternatively, in some embodiments, a mixture of both ligands and compounds may be present together in solution.

The term “compound,” “target,” or “target compound” as used herein refers to a biological molecule, biochemical or chemical entity, molecule, or pharmaceutical drug candidate to be detected.

The term “biological molecule” or “biomolecule” refers to any kind of biological entity, including, such as, modified and unmodified nucleotides, nucleosides, peptides, polypeptides, proteins, lipids, or saccharides.

The term “cognate,” “corresponding,” or “paired” refers to the reciprocal moiety of a molecule to another; in particular, a ligand that can bind specifically to a given receptor is called a ligand-receptor pair.

The term “biospot” or “microspot” refers to a discrete or defined area, locus, or spot on the surface of a substrate, containing a biological or chemical probe.

The term “receptor microspot” refers to a microspot containing a deposit of membrane-bound proteins. The receptor may include a GPCR, a ligand-gated ion channel receptor, a tyrosine kinase receptor, serine/threonine kinase receptor, or guanylate cyclase receptor.

The term “GPCR” refers to a guanine nucleotide-binding protein-coupled receptor. The GPCR can have either a natural or modified sequence.

The term “GPCR membrane” or “GPCR membrane fragment” refers to a biological membrane or cell membrane fragment having a GPCR embedded within a membrane layer, or a micelle having a GPCR reconstituted within the micelle.

The term “GPCR microspot” refers to a microspot containing a deposit of G-protein coupled receptors (GPCRs). The corresponding microspots are referred to as “probe microspots,” and these microspots are arranged in a spatially addressable manner to form a microarray.

The term “probe” or “receptor probe” refers to a receptor molecule (e.g., GPCR), which according to the nomenclature recommended by B. Phimister (Nature Genetics 1999, 21 supplement, pp. 1-60.), is immobilized to a substrate surface. Preferably, probes are arranged in a spatially addressable manner to form an array of microspots. When the array is exposed to a sample of interest, molecules in the sample selectively and specifically binds to their binding partners (i.e., probes). The binding of a “target” to the probes occurs to an extent determined by the concentration of that “target” molecule and its affinity for a particular probe.

The term “substrate” or “substrate surface” as used herein refers to a solid or semi-solid, or porous material (e.g., micro- or nano-scale pores), which can form a stable support. The substrate surface can be selected from a variety of materials, including for instance, glass, ceramic, metals, polymers, plastics, or combinations of these.

The term “functionalization” as used herein relates to modification of a solid substrate to provide a plurality of functional groups on the substrate surface. The phrase “functionalized surface” as used herein refers to a substrate surface that has been modified to have a plurality of functional groups present thereon. The surface may have an amine-presenting functionality (e.g., γ-amino-propylsilane (GAPS) coating), or may be coated with amine presenting polymers such as chitosan and poly(ethyleneimine).

### Section II—Description

Previously, we have demonstrated that one may fabricate GPCR microarrays using conventional robotic pin printing technologies and cell membrane preparations containing GPCRs from a cell line over-expressing the receptor. (See e.g., U.S. patent application Ser. Nos. 09/974,415 (U.S. Patent Publication No. 2002/0019015 A1), and 09/854,786, (U.S. Patent Publication No. 2002/0094544 A1) the contents of which are incorporated herein by reference). Also, we have described certain methods for fabricating biological membrane microarrays on substrates presenting certain surface chemistries. (See, U.S. patent application Ser. No. 10/300,954, incorporated herein by reference.) These kinds of arrays can be prepared under ambient conditions (i.e., not in fluid or wet conditions), stored at about 4° C., and still retain their functionality for an extended period of time thereafter.

Although such arrays are useful for compound profiling and screening, the potential for multiplexed applications have not been easily or readily achievable. Even though GPCR microarrays conceptually are suited to analyzing multiple GPCRs simultaneously, a number of issues or associated problems have stood in the way of making this concept a reality. First, unlike DNA hybridization using DNA microarrays, the interaction between GPCRs and ligands is much more complicated. Ligands for GPCRs are very diverse, including biogenic amines, peptides and proteins, lipids, nucleotides, excitatory amino acids and ions, small chemical compounds, etc. (Morris, A. J., and Malbon, C. C., “Physiological Regulation of G-Protein-linked Signaling,” Physiol. Rev. 1999, 79, 1373-1430.) A particular GPCR could couple to one or more trimeric G proteins in a cell line. The binding affinities of agonists to a GPCR depend on the coupling state of the receptor with its G proteins. Compounds that bind with a receptor might have different functionalities, such as agonism, antagonism, super-agonism or inverse agonism. The binding sites involved might be different for different compounds binding to the same receptor.

Second, for assay development, buffer compatibility and optimization should be also considered. For example, some GPCR-ligand interactions depend strongly on the presence of particular divalent cations such as Mg²⁺ or Mn²⁺. The buffer composition can not only affect the functionality of the membrane proteins, but also can affect the binding affinity of ligands or compounds to the receptors in arrays. In addition, the buffer composition also may have a negative impact on the stability and packing of receptor-containing lipid membranes immobilized on the surface, therefore decreasing array performance and assay robustness.

Third, the choice of labeled ligands is equally, if not more critical. The ideal labeled-ligand should bind only to its corresponding receptor with high affinity, and with minimal cross reactivity with other receptors in the same microarray, so as to enable the use of the labeled-ligand at low concentrations and to permit stringent washing processes. High affinity, however, is not a requirement for real-time fluorescence measurements. Empirical data suggest that ideal fluorescent ligands for GPCR microarray applications should have specific properties, such as being relatively hydrophilic, having low net charge(s) (preferably, they are positively charged or neutral), good photostability, high binding affinity (low K_{d}, preferably within a nanomolar range of ˜0.5 nM to ˜10 nM), high specificity (≧50% or 60%) for a given receptor in the array, and minimal cross-talk to other receptors in the same microarray.

Unlike with nucleotide microarrays (e.g. labeling RNA with dye-phophoramidite for producing labeled cDNA by reverse transcription), no format can be applied as a universal solution for the design of cocktails of labeled ligands for protein microarrays. The lack of a simple code analogous to DNA-base pairing for receptor-ligand interactions has been a deterrent to the realization of protein microarrays for highly multiplexed compound profiling. Setting the basic guidelines for specificity and cross-reactivity, and employing upfront rigor to the selection process can enable the practical use of protein microarrays for compound screening.

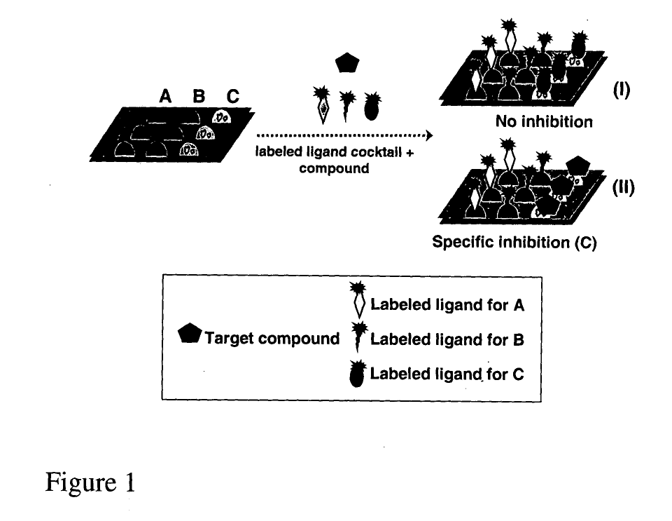

To address these issues and to achieve consistently reliable multiplexed binding assays, we propose the present invention, which meets and incorporates the foregoing criteria. As an illustration of the general concept of the invention, FIG. 1 presents a schematic representation of a multiplexed competitive binding assay that uses a cocktail solution of labeled ligands with a GPCR microarray.

In FIG. 1, three kinds of receptors, one in each column of microspots, are immobilized on a support surface in an array. To this array is applied a “cocktail” solution containing three labeled-ligands with a test compound. The ligands A and B, corresponding to receptors A and B, are labeled using fluorophores that are either identical or similar in terms of their spectral fluorescence signature. The ligand corresponding to receptor C is labeled with a different kind of fluorophore that exhibits an excitation and emission spectrum distinguishable from the fluorophore(s) labeling ligands A and B. Once the cocktail solution is applied, two possible situations are depicted. In the first situation, no specific inhibition is observed since the test compound does not bind to any of the receptor proteins. In the second situation, we observe a reduction in signal intensity due to specific inhibition from the test compound to the binding of the paired labeled-ligand to its cognate receptor C. Thus, the test compound specifically inhibits binding to receptor C.

The benefits of the present invention encourage multiplexing and miniaturization, which are particularly pertinent for today, due to the increased pace and need for rapid target identification. Obtaining binding information against multiple receptors is inherently powerful; with the present invention multidimensional structure-activity relationships can now be achieved, such as multiple compounds against a single receptor and multiple compounds against multiple receptors.

Binding assays generally are directed to protein/peptide profiling uses. Current examples of such assays involve immobilized protein molecules of interest on a surface at defined locations. (See, Wilson, D. S. and Nock, S., “Functional Protein Microarrays,” Curr. Opinion in Chemical Biology 2001, 6, 81-85; Zhu, H., et al., “Global Analysis of Protein Activities Using Proteome Chips,” Science 2001, 293, 1201-2105.) Arrays of antibody probes also have been used for protein profiling, to measure protein abundances in blood, to measure cytokine abundances, as well as to capture leukocytes/phenotyping leukemias. Arrays of antigen probes have been used for reverse immunoassay to measure auto-immune antibodies and allergies.

Others have also suggested methods and apparatus for sequencing, fingerprinting, and mapping biological macromolecules, typically biological polymers. For instance, U.S. Pat. No. 6,416,952 B1, issued to Pirrung et al., involves using a plurality of polypeptides with known sequence to detect with fluorescently labeled polypeptide targets. The methods developed by Pirrung et al. have not taken account the complexity associated with multiplexed applications as outlined above, and likely are not feasible for such purposes. Further, their platform is mainly constructed for a direct binding profile assay of a peptide/protein in a sample. In contrast, the present invention involves a indirect binding assay, which takes advantage of the competitive ability of small biological, biochemical or chemical molecules or compounds (e.g., molecular weight ≦˜10,000 daltons, preferably between about 100-5000 daltons) to bind with receptors on the array against the predetermined labeled-ligands's ability to bind with the same receptors. Moreover, Pirrung et al.'s platform relies on a design in which the number of the probe polypeptide(s) in the microarray overwhelms the number of available target polypeptide molecules to be detected in a sample. Conversely, the present invention takes advantage of a reversible binding system in which the labeled ligands binds to their paired probe receptors in the microarray. In particular, the probe receptors can be, and are preferably (profiling or screening tests), saturated by the binding of labeled ligands.

From a combinatorial “numbers” perspective, attempting to map out biological target space for a group of proteins (e.g. ˜700 GPCRs) is actually more tractable than attempting to map the vastness of chemical ligand space. By greatly increasing the potential to match this key target type with compound libraries, we anticipate that GPCR microarrays will emerge as an important tool for drug discovery and basic research.

I. Thematic GPCR Microarrays

Unlike tens of thousands of genes one has to consider for array design in DNA microarrays, there is much smaller number of GPCRs—the human genome contains about 400-700 GPCRs. Scientitist have discovered ligands associated with about 200 GPCRs. Receptors whose natural agonist remains unidentified are referred to as “orphan” GPCRs. In one embodiment, all of the known and/or orphan GPCRs could be arrayed on a surface to form a full index array; such arrays can be used for target identification, natural agonist identification and compound screening. In another embodiment, one subfamily member of each subfamily of all GPCRs can be arrayed together on a surface to form a representative index array; such arrays can be used for compound screening, and more suitable for classifying compounds against family GPCRs.

The selectivity of a potential drug compound to a targeted GPCR versus other GPCRs in the same organ, tissue or even single cell is an extremely important factor to be considered and monitored during drug development. Currently almost all HTS techniques are related to single target screening at one time. GPCR arrays can be used to evaluate the selectivity of multiple compounds of interest to a variety of receptors. In an embodiment of the present invention, members of a single or several related subfamilies of GPCRs can be arrayed on a surface; such arrays are preferably for compound pharmacological profiling and selectivity screening.

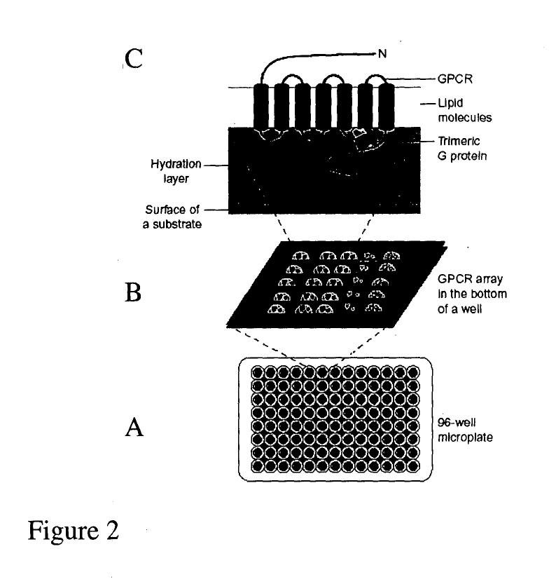

FIGS. 2A-C show an enlarged, exploded view of the bottom of microtiter well plate (a.k.a., microplate). FIG. 2A is the overview of the microplate. FIG. 2B depicts a small set of pre-selected GPCRs associated, preferably fluidly, or embedded in lipid membranes. The membranes are deposited and immobilized at defined locations on the bottom surface of a well in the microplate forming an array of microspots. FIG. 2C illustrates, as an idea orientation, the lipid-membrane containing the GPCR is immobilized and arranged with the N-terminal-side of the receptor protein molecule directed away from the solid support of the well-bottom. Preferably, the GPCR molecules in the membranes are coupled with certain trimeric G-protein.

In a preferred embodiment, GPCRs that share similar specific tissue distribution, or specific roles in physiology and pharmacology are arrayed on a surface; such arrays are preferred for compound selectivity screening. Some GPCRs are preferably distributed in certain types of tissues. For example, some receptors including the muscarinic acetylcholine receptor, dopamine 2 receptor, histamine 2 receptor, serotonin 4 receptor and prostaglandin receptor prominently distribute in the gastrointestinal system, while some receptors including serotonin 1A/1D and 2A/2C receptor, neurotensin 1 and 2 receptors, opioid receptors (mu, delta, kappa, ORL-1), and dopamine 2/3 receptors prominently distribute in the central nervous system (Stadel, J. M., et al. TIPS 1997, v. 18, 430-437). Likewise, some receptors are associated with known physiological and pharmacological functions. For example, certain GPCRs for chemokines act as co-factors for HIV infection (Feng, Y., et al., Science 1996, v. 272, 872-876; Deng, H. K., et al. Nature 1996, v. 381, 661-666). Additionally, some receptors including serotonin 1A, adenosine A 1/2 A and angiotensin receptors play an important role in anxiety and hypertension, while some receptors including opioid receptors, calcitonin gene-related peptide receptors and neuropeptide FF receptors are related to pain control.

In another preferably embodiment, a GPCR and its physiologically important or random mutants are arrayed on a surface; such arrays are preferably suitable for studying structure-activity relationships, as well as screening highly specific drug compounds for a GPCR mutant that plays a key role in a given human disease or cancer. Some GPCRs and their mutants are related to the development of certain tumors. For example, some mutations of rhodopsin are related to retinitis pigmentosa, while some mutations of vasopressin V2 are related to X-linked nephrogenic diabetes (Stadel, J. M., et al., Trends in Pharamaco. Sciences 1997, v. 18, 430-437).

In an alternative embodiment, the GPCRs of personal interest can be arrayed on a surface; such custom arrays can be used to serve their own selectivity screening of compounds.

II. Labeled Ligand

In terms of functionality relative to their paired GPCRs, the ligand can be selected from a group including: antagonists, agonists, partial-agonists, or inverse-agonists. The ligand also may be a naturally occurring agonist or a synthetic chemical, biochemical, or biological compound or molecule. In terms of chemical identity or structure, the ligand can be a nucleotide, a nucleoside, a modified nucleotide or nucleoside, an organic or inorganic compound, a peptide, a polypeptide or protein, a lipid, or a modified lipid.

For multiplexed binding assays, the ligand should bind readily to a corresponding receptor protein and may be labeled. According to an embodiment, when it is labeled, the ligand should be detectable by means of a variety of state-of-the-art techniques or methodologies. The label is selected based on the desired detection technology employed, including fluorescence, radioactive detection, chemical or bioluminescence, or phosphor up-conversion, or other technologies. Preferably, the label is a fluorescent dye (e.g., Bodipy-fluorescein, Bodipy-TMR., Rhodamine, Texas-Red, Cy-3, Cy-5, or fluorescein). Alternatively, the label is a radio-isotope, such as tritium or P³², S³⁵, or I¹²⁵. For another label species, such as biotin, the detection technique can be a labeled streptavidin, anti-biotin antibody, or a streptavidin or anti-biotin antibody-coated particle (e.g., gold nanoparticle, wherein the resonant light scattering may be employed for detection).

In a second embodiment, a direct-binding assay based on an evanescent-field detector employing a grating-coupled waveguide, surface-plasmon resonance, or other mass-based biosensor system, can be used. In this situation, the ligand can be either unlabeled or labeled. Any labeling species may be included for direct binding assays. In particular embodiments, a label can be a species that may have a sufficient mass or an optical charactertistic useful for enhanced detection sensitivity. For instance, a gold particle with a diameter of about 1 nanometer to about 45 nanometers, or up to about 100 nanometers, can be attached to a ligand.

For the present multiplexed binding assay to work effectively, a labeled ligand should satisfy several critical properties, as mentioned above. An ideal fluorescently labeled ligand for GPCR microarray applications should be relatively hydrophilic, have low net charge, have good photostability, and have a binding affinity (K_{d}) of several nanomolar with a specificity of greater than or equal to about 55% or 60% for its paired receptor in the array. The ligand should also exhibit minimal cross-activity to other kinds of receptors.

III. Labeled-Ligand Cocktail Solution

Generally, according to the invention, a cocktail solution containing more than one labeled ligand is used in multiplexed binding assays. In the cocktail solution, each labeled ligand should bind only to its paired GPCR(s) in an array. For example, Cy5-naltrexone works as a labeled ligand for delta2 opioid receptor, because Cy5-naltrexone is more hydrophilic than fluorescein-naltrexone (e.g., commercially available from the Molecular Probes, Inc.), can bind to delta2 opioid receptor with a K_{d} of 2.5 nM with a specificity of ˜90% at the concentration of one to four fold (1-4×) of K_{d}, and has minimal cross activity to a number of receptors, such as neurotensin receptor (NTR1), neurokinin receptor subtype 2 (NK2), motilin receptor (MOTR), and beta1, beta2, and alpha2A adrenergic receptors. Similarly, Bodipy-TMR-motilin 1-16 (BT-MOT16) is chosen as a labeled ligand for motilin receptor, because BT-MOT16 is relatively hydrophilic and has neutral charge at physiological pH, can bind to the MOTR with a K_{d} of 3 nM with a specificity of 75% at the concentration of 1-4× K_{d}, and has minimal cross activity to a number of receptor, such as neurotensin receptor (NTR1), delta2 opioid reeceptor, neurokinin receptor subtype 2 (NK2), and beta1, beta2, and alpha2A adrenergic receptors.

Alternatively, a single type of labeled ligand could be employed for several different receptors if the ligand binds to these receptors with the desired affinity and specificity. For example, Bodipy-TMR-CGP 12177 has been reported to bind specifically to the β1 and β2 adrenergic receptors with similar affinity (K_{d} of 1-2 nM). (Fang, Y., et al., J. Am. Chem. Soc. 2002, 124, 2394-2395; Baker, J. G., et al., Brit. J. Pharmacol. 2002, 139, 232-242.)

For multiplexed binding assays, it is not necessary that the labeled ligands have similar intensities after binding to their respective receptors on the microarray. Analysis of the data for each receptor as a function of the presence of different compounds or compound concentrations can be done in several ways including absolute signal intensity, relative signal intensity, or a ratiometric analysis if two different dyes are used. Labeled ligands in the cocktail could be agonists and/or antagonists; the concentration of each ligand in the cocktail should be relatively close to the value of the K_{d} of the ligand to the paired receptor(s) in order to maximize total binding signal(s) as well as specificity. Preferably, the concentration of labeled ligand can be about ˜0.7 or 1 to 4 fold of the K_{d} value. Labeled ligands in the cocktail can be labeled with different fluorescent dye moieties (e.g., rhodamine-, Bodipy-TMR-, Cy3-, Cy5-, etc); the detection can be multi-channel fluorescence scanner (e.g, FITC, Cy3, TR, Cy5 channels). Overall, multiplexed binding assays require buffer conditions that are “universal” and labeled ligand cocktails in which each ligand maintains its cognate specificity in the presence of additional ligands and receptors. These requirements demand a systematic investigation of universal binding conditions and a careful consideration of the appropriate fluorescent label, the cognate ligand for label attachment, and the chemistry of labeling.

IV. Methods of Use

Although the examples presented herein employ fluorescence-based detection because of the availability of fluorescence microarray scanners (e.g., Genepix), other kinds of detection also may be used. For instance, radioactivity based detection, however, should be feasible using high resolution phosphorimagers (e.g., the Typhoon 9410, Amersham Biosciences), and may even provide superior data relative to fluorescence based detection because of reduced issues with non-specific binding.

1. Multiplexed Saturation Assay for Parallel K_{d} Determination

Multiplexed saturation assay can be used to determine binding constants (i.e., K_{d}) of labeled ligands to their paired receptors in parallel. In this assay, a cocktail of labeled ligands are used; each labeled ligand binds specifically to its own paired GPCR in the arrays with no or minimal cross activity to other receptors in the same arrays. Two subsets of microarrays were incubated individually with a buffered solution containing labeled ligand cocktail, each at different concentrations in the absence and presence of their unlabeled counterpart ligand in excess. We determined the amount of specific binding by subtracting the fluorescence signals of the first set of arrays from the second set of arrays incubated with the labeled ligands (at the same concentration) and excess unlabeled ligand. Scatchard analysis was used to estimate the K_{d}.

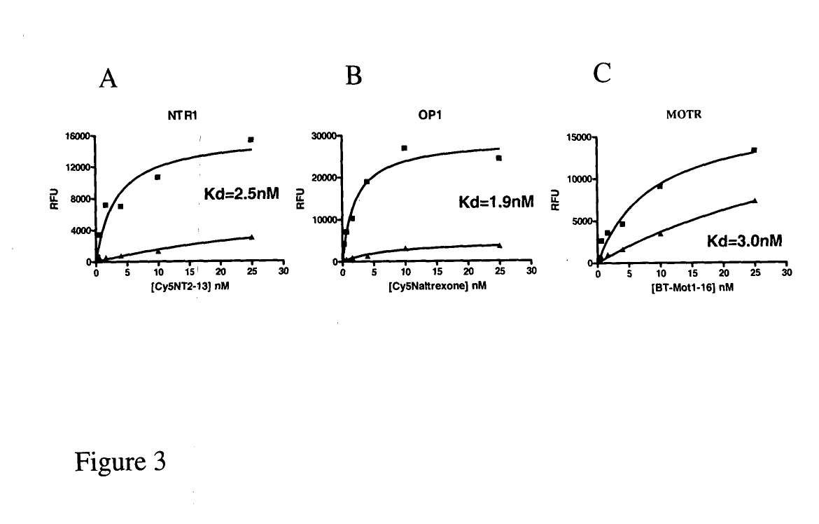

For an example, the binding of BT-motilin 1-16 (BT-MOT16), Cy5-neurotensin 2-13 (Cy5-NT), and Cy5-naltrexone to an microarray consisting of human motilin receptor (MOTR), neurotensin receptor subtype 1 (NTR1) and delta 2/opioid receptor (62, OP1) is shown in FIG. 3. The plot in FIG. 3A shows the binding curve of a labeled ligand reacting with the NTR1; FIG. 3B shows the reaction of a labeled ligand with the OP1; and, FIG. 3C shows the binding curve of a labeled ligand with MOTR. The reactions of FIGS. 3A and 3B are monitored in Cy5 channel, whereas FIG. 3C is obtained in Cy3 channel.

To estimate a K_{d} value for each ligand to its paired receptor, a microarray having receptors (OP1, MOTR, and NTR1) is treated with a cocktail solution (Cy5-NaI, BT-Mot, and cy5-NT, each at concentration of 0.26, 0.64, 1.6, 4, 10, and 25 nM), in the absence or presence of excess unlabeled ligands (MOT, naltrexone, NT, each at a concentration of ˜2 μM). Specific binding was determined by subtracting the signal in the presence of the cocktail alone from the signal obtained from a solution also containing unlabeled ligands in excess. The K_{d} value is calculated using a computer program (e.g., Prism™).

2. Multiplexed Competitive Binding Assays for Selective Potency of a Single Compound

Multiplexed competitive binding assays can be used to determine “selective potency” of a single compound against each labeled ligand in the cocktail to its own paired GPCR in the microarrays. In this assay, a cocktail of labeled ligands is used; each labeled ligand binds specifically to its own paired GPCR in the arrays with no or minimal cross activity to other receptors in the same arrays. Moreover, multiple sub-arrays are treated individually with a given compound at different concentrations in the presence of the labeled ligand cocktail, each at a predetermined, constant concentration. The fluorescence intensity of each receptor microspot was examined as a function of the concentration of the compound, and an IC₅₀ value was later extracted.

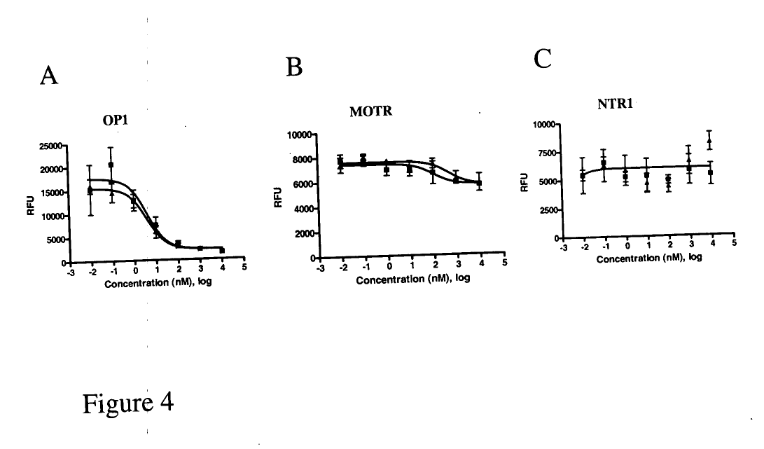

The selective potencies of a single compound, naltrexone, against the binding of Cy5-naltrexone to delta2, Cy5-NT to NTR1, and BT-MOT16 to MOTR were examined using a microarray consisting of MOTR, NTR1 and delta2. FIGS. 4A-C represent the results of an experiment conducted according to an embodiment of present method. FIG. 4A shows concentration dependent inhibition profile of the binding reaction between Cy5-naltrexone and OP1-receptor by naltrexone. FIGS. 4B and 4C, respectively, show the concentration dependent inhibition profiles of the binding reaction of BT-MOT16 with MOTR, and Cy5-NT with NTR1. Each figure shows data derived from duplicate experiments. The concentration of each of the three labeled ligands in the cocktail solution is: BT-MOT16, 5 nM; Cy5-NT, 5 nM; and Cy5-naltrexone, 2.5 nM.

3. Multiplexed Competitive Binding Assays for Relative Potency of Multiple Compounds

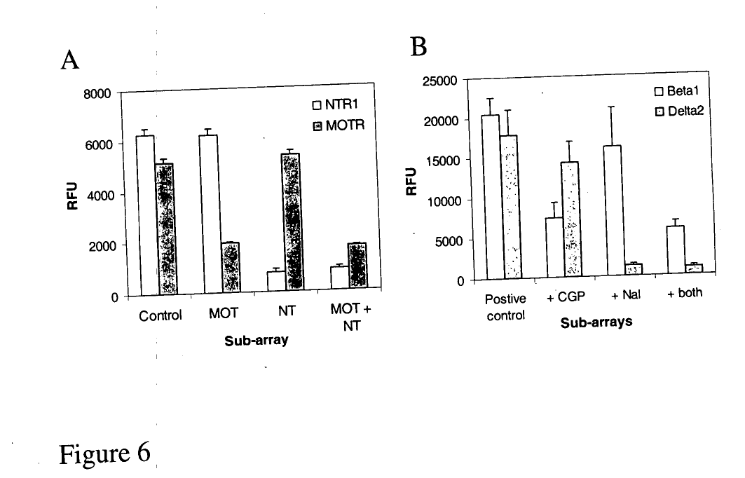

Multiplexed competitive binding assays can be used to determine relative potency of multiple compounds against the binding of each labeled ligand in the cocktail solution to its paired corresponding GPCR in a microarray. In such an assay, one can pre-determine that each compound binds specifically to its own receptor within a concentration range employed (e.g., up to micromolar concentrations, using alternative methods or by using an initial screen using GPCR microarrays, as illustrated in FIG. 6) with minimal cross-binding to other receptors in the same array. As before, a cocktail solution of labeled ligands is used. Each labeled ligand binds specifically to its own paired GPCR in the array with little or no cross activity to other receptors in the same arrays. The sub-arrays are treated separately and individually with a solution of unlabeled compounds at different concentrations in the presence of the cocktail solution of labeled ligands, each of which are at a fixed concentration. The fluorescence intensity of each receptor microspot is examined as a function of the concentration of the cocktail of compounds, and IC50 values for the compounds are estimated. Two sets of experiments are carried out. The first set involves using three compounds (neurotensin for NTR1, motilin for MOTR, and naltrexone for delta2). The second set involves using three other compounds (neuromedin N to NTR1, endomorphin II to delta2, motilin for MOTR).

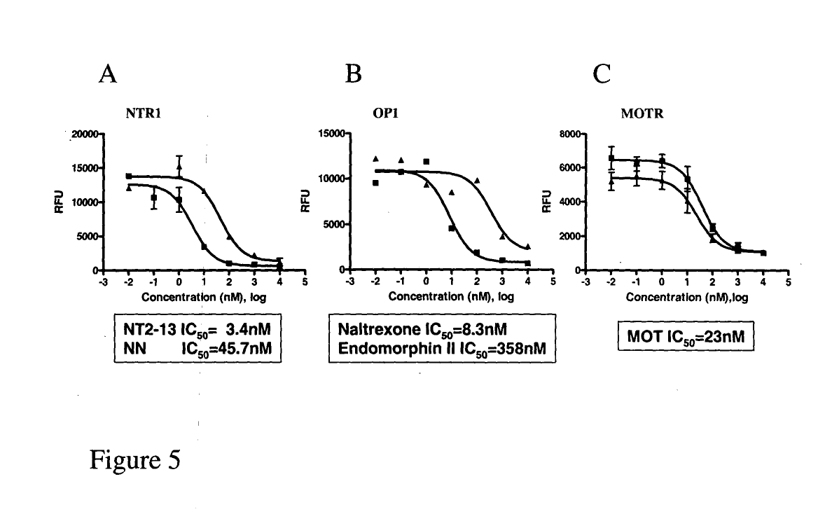

Using a microarray with MOTR, NTR1 and delta2, the relative potencies of these compounds against the binding affinity of Cy5-naltrexone to delta2, Cy5-NT to NTR1, and BT-MOT16 to MOTR are examined. The results are summarized graphically in accompanying FIGS. 5A-C. Multiplexed estimations of IC50 values obtained by treating a microarray consisting of MOT, NTR1, and OP1 receptors treated with a cocktail of labeled ligands (cy5-NaI, BT-mot, cy5-NT) and a mixture of unlabeled ligands. The mixture of unlabeled ligands contained neurotensin, motilin, and naltrexone or neuromedin N, motilin, and endomorphin II. From previous experiments, it was determined that each ligand binds to only one of the receptors in the GPCR microarray. The plots for the mixtures are grouped for each receptor to highlight their differential affinities. Two plots are shown for inhibition by motilin since it was part of both mixtures.

4. Multiplexed Competitive Binding Assays for Compound Screening Using Cocktail Solution of Labeled-Ligands.

Multiplexed competitive binding assays can be used for screening compounds which potentially can inhibit the binding of at least one labeled ligand in the cocktail solution with their paired GPCRs. According to such an assay, a cocktail solution of labeled ligands is provided. Each labeled ligand binds specifically to its own corresponding GPCR in the arrays with minimal or no cross activity with other receptors in the same array. Furthermore, each sub-array is treated individually with at lease one compound at a fixed concentration (generally ˜1 μM) in the presence of a fixed concentration of the cocktail solution of labeled ligands. The results for two examples according to the invention for screening compounds against microarrays having MOTR and NTR1 probes, and another consisting of delta2 and beta1 receptors, are presented in FIG. 6. The results show that the binding of fluorescent ligands with their corresponding receptors can be specifically inhibited only by their own known ligands.

5. Multiplexed Competitive Binding Assays for Compound Screening Using a Single Labeled-Ligand.

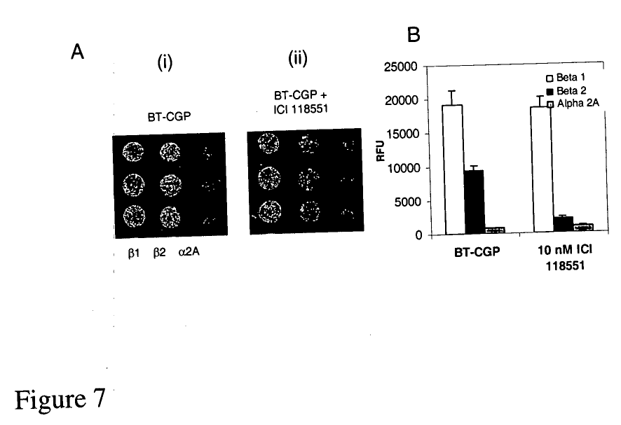

According to a simplified version of multiplexed binding assays for compound screening, one may employ a single labeled ligand that can specifically bind with at lease two receptors in the same sub-arrays with desired affinities. In one example, for compound selectivity screening, one may use microarrays having three GPCR members selected from the adrenergic receptor subfamily (β1, β2, and α_{2A}) Fluorescently labeled CGP 12177 ligand molecules can bind with β1 and β2 in the arrays, hence this kind of ligand are used to monitor the two receptors. Results suggest that ICI 118 551, a drug compound targeted towards adrenergic receptors, has significantly higher affinity for β2 (Kᵢ of 4.8 nM) over β1 (Kᵢ of 80 nM). These findings are consistent with data reported in the relevant scientific literature. In conjunction with data from FIGS. 3-7, a process of using labeled ligand cocktails, in which one or more of the labeled ligands are specifically designed to bind to more than one receptor (e.g. a labeled ligand per receptor subfamily) is also suggested.

6. Multiplexed Displacement Assays for Compound Screening

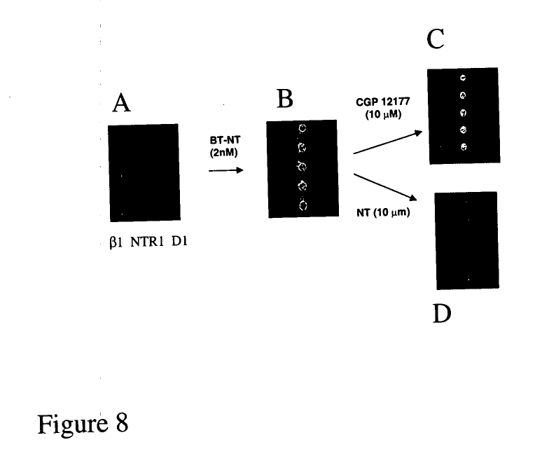

An alternative multiplexed binding assay for compound screening involves using fluorescently labeled ligands that are pre-bound to GPCR molecules. An assay of this format involves incubating the GPCR microarray with the labeled ligands prior to treatment with solutions containing putative ligands. This kind of assay is feasible because of the high affinity of labeled ligands to their paired GPCRs. The binding event is essentially irreversible over the duration of the assay, since mass transport limited rebinding greatly reduces the rate of dissociation of labeled ligands. Treatment of the array with solutions containing a competitive ligand, however, can effect the displacement of a bound ligand. Data for an example is summarized in the images of FIGS. 8A-D.

V. Increased Multiplexing Capability

The present invention also describes a method for increasing the overall multiplexing capability of a microarray. Such a method can be particularly useful for microarrays located in a microtiter plate, which have multiple biological probes arrayed onto the bottom of either 96-well or 384-well microtiter plates. Due to the spatial constraints on the bottom surface of a microplate well, only a limited number of biological probes may be immobilized and analyzed at one time. For example, in a 384-well plate only 0.136 cm² is available for depositing an array. One can easily calculate that each well in 384-well microtiter plate can only be used to array 3-4 receptors, each in triplicate; or for a maximum of about 20 elements per well. This limited number of elements is typically insufficient for high throughput screening uses, which have a selectivity panel of probe receptors, ideally numbering in the range of about 5-10, with each kind of probe in the array in triplicate or quadruplicate, for statistical reliability.

The method involves the use of at least two receptors co-immobilized within a single microspot, in combination with at least a two-color detection approach enabling at least a doubling of the capacity of probe elements for biological microarrays. Although the new approach is described in terms of GPCR microarrays, the general concept is not necessarily limited to just one species or kind of microarray application. Any type of biological microarray which uses probe elements can benefit from the present method. In particular embodiments of the invention, at least two biological targets are mixed together and the combined mixture is used to fabricate microarrays. The resulting microarrays have a minimal two fold capacity for multiplexing.

In another embodiment, the method exploits a system with at least two-color detection. A two-color detection system, for example, employs a cocktail of two mixed labeled-ligands with different label tags, and each labeled ligand specifically binds to only one probe in each microspot. The tags can be different in terms of physical and chemical properties. Preferably, these tags are fluorescence dyes. In another embodiment these tags are nano-particles that give rise to different mass or light emitting (scattering) properties. Since most current fluorescence imaging technologies (e.g., FITC, Cy3, Texas Red, and Cy5) are limited to four channels, four probes can be set in each microspot. This number of probes, however, is not necessarily limiting of the present method. As technology advances for microarray applications, a greater number of probes may be incorporated per microspot.

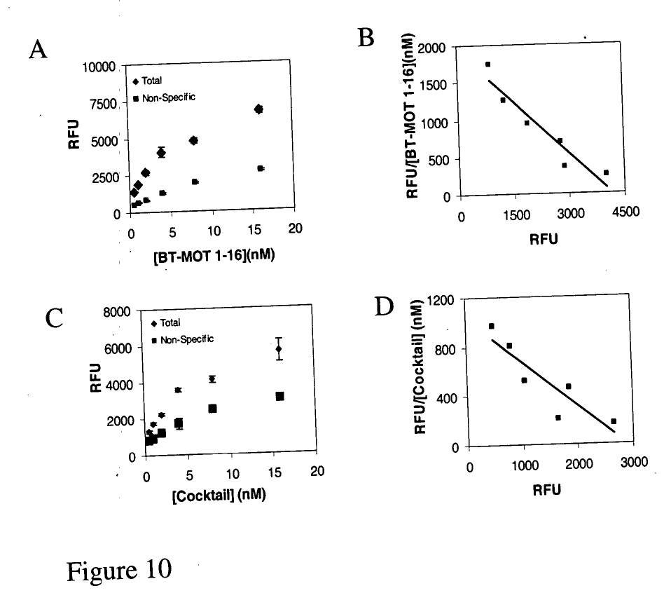

Proof-of-the concept performance of this approach has been demonstrated as shown in FIG. 10 enclosed. Advantages of this invention include the ability to increase the multiplexing capacity of microarrays in microplate by as much as about 200% (i.e., can be theoretically extended to 4 fold), and reduce the printing and assay variability between two receptors because these two receptors are mixed prior to printing, while experiencing no significant reduction in assay sensitivity, no significant alteration in pharmacological properties of ligands binding to the receptors in the arrays, although the maximum binding sites in each microspot decreases due to the mixing of two receptors.

Hence, to reiterate, the present invention relates to a thematic GPCR microarray and methods for profiling or screening ligands or compounds. The thematic multiplexed GPCR microarray comprises: a plurality of GPCRs arranged on a substrate at positionally defined locations; said GPCRs are either a) a member from each subfamily of GPCRs, or b) at least a single member selected from several related subfamilies of GPCRs, or c) a GPCR and its mutants or its corresponding GPCRs originated from different species. The GPCRs in said microarray are related to each other by either: functionality, physiology, family, pathology, tissue-distribution, or mutagenesis, or evolutionary history.

According to an embodiment, the method comprises: a) providing an plurality of receptor microspots on a substrate to form an array; b) preparing a cocktail solution of labeled ligands, each labeled ligand having an affinity to bind with at least one corresponding paired receptor in said array; wherein said cocktail solution contains either 1) only labeled ligands, each at a different concentration, or 2) labeled ligands, each at a different concentration, in the presence of counterpart ligands in excess; c) contacting said cocktail solution with said array; and d) determining the binding affinity of each labeled ligand to its said paired receptor. The receptor is a guanine nucleotide-binding protein-coupled receptor (GPCR), a ligand-gated ion channel receptor, a tyrosine kinase receptor, serine/threonine kinase receptor, or guanylate cyclase receptor. The GPCR is associated with a biological membrane, which may be either i) a cell membrane fragment containing a GPCR, or ii) a liposome or micell containing a reconstituted GPCR. The labeled-ligand in said cocktail solution is a chemical molecule or biological molecule that can bind readily to a receptor with a specific binding affinity constant.

In another embodiment, a method profiling or screening a compound or target comprises: a) providing an plurality of receptor microspots on a solid surface to form an array; b) preparing a cocktail solution of labeled ligands, each labeled ligand having an affinity to bind with at least one corresponding paired receptor in said array; wherein a compound or multiple compounds are either present or absent from said cocktail solution; c) contacting said cocktail solution with said array; and d) determining the potency of said compound against the binding of each labeled ligand to its said paired receptor.

Alternatively, the method comprises: a) providing an plurality of receptor microspots on a solid surface to form an array; b) preparing a cocktail solution of labeled ligands, each labeled ligand having an affinity to bind with at least one corresponding paired receptor in said array; c) contacting said cocktail solution with said array; d) determining the total binding signal of each receptor; e) sequentially contacting said array with a second solution containing a compound; and f) determining the amount of pre-bound labeled ligands to each receptor displaced by said compound.

To screen or profile a compound using a multiplexed binding assay, the method comprises: a) providing an plurality of GPCR microspots on a solid surface to form an array; b) preparing a cocktail solution of labeled ligands, each labeled ligand having an affinity to bind with at least one corresponding paired GPCR in said array, in either the absence or presence of a compound; c) contacting said cocktail solution with said array;
and d) determining the binding profile of the compound against the labeled ligand to its said paired receptor.

In another embodiment, the method to screen or profile a compound comprises: a) providing an plurality of receptor microspots on a solid surface to form an array; b) preparing a solution containing at least one compound; c) contacting the array with the solution; d) detecting the binding of the compound to said receptors; and c) determining the binding profile of said compound to its said paired receptor.

The present invention has been described both in general and in detail by way of examples. Persons skilled in the art will understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Hence, unless changes otherwise depart from the scope of the invention, the changes should be construed as being included herein.

1. A method to profile or screen target compounds using a microarray, the method comprising: a) providing an plurality of receptor microspots on a substrate to form an array; b) preparing a cocktail solution of labeled ligands, each labeled ligand having an affinity to bind with at least one corresponding paired receptor in said array; wherein said cocktail solution contains either 1) only labeled ligands, each at a predetermined concentration, or 2) labeled ligands, each at a predetermined concentration, in the presence of a target compound; c) contacting said cocktail solution with said array; and d) determining the binding profile of each labeled ligand or said target compound to its respective paired receptor.

2. The method according to claim 1, wherein the receptor is a membrane-bound protein.

3. The method according to claim 1, wherein said receptor is a guanine nucleotide-binding protein-coupled receptor (GPCR), a ligand-gated ion channel receptor, a tyrosine kinase receptor, serine/threonine kinase receptor, or guanylate-cyclase receptor.

4. The method according to claim 3, wherein each set of said GPCRs in said microarray are related to each other by either functionality, physiology, family, pathology, tissue-distribution, mutagenesis, or evolutionary history.

5. The method according to claim 3, wherein said GPCR is associated with a biological membrane.

6. The method according to claim 5, wherein said biological membrane is either i) a cell membrane fragment containing a GPCR, or ii) a liposome or micelle containing a reconstituted GPCR.

7. The method according to claim 1, wherein said labeled-ligand in said cocktail solution is a chemical molecule or biological molecule that can bind readily to a receptor with a specific binding affinity constant.

8. The method according to claim 7, wherein said labeled ligand is labeled with one of the following: a fluorescent moiety, a radioactive isotope, a hapten molecule, or gold nanoparticle label.

9. The method according to claim 1, wherein said cocktail solution contains an assembly of labeled ligands in a binding buffer; each ligand having a propensity to bind specifically to at least one receptor in said microarray.

10. The method according to claim 1, wherein said substrate has a functionalized surface.

11. The method according to claim 10, wherein said substrate is made from a solid or semi-solid, or porous material.

12. The method according to claim 11, wherein said porous material has either micro- or nano-scale pores.

13. The method according to claim 10, wherein said substrate surface is an amine presenting surface.

14. The method according to claim 13, wherein said amine-presenting surface is coated with a γ-amino-propylsilane (GAPS).

15. The method according to claim 13, wherein said amine-presenting surface is coated with amine-presenting polymers, chitosan, or poly(ethyleneimine).

16. The method according to claim 1, wherein said method involves providing at least two receptors co-immobilized within a single microspot, in combination with a two-color detection technique, to at least double the capacity of probe elements.

17. The method according to claim 16, wherein said method involves providing four or more receptors co-immobilized within a single microspot, in combination with a four or more color detection technique, to at least double the capacity of probe elements.

18. A method to determine binding affinity of labeled ligands using microarrays, the method comprising: a) providing an plurality of receptor microspots on a substrate to form an array; b) preparing a cocktail solution of labeled ligands, each labeled ligand having an affinity to bind with at least one corresponding paired receptor in said array; wherein said cocktail solution contains either 1) only labeled ligands, each at a different concentration, or 2) labeled ligands, each at a different concentration, in the presence of counterpart ligands in excess; c) contacting said cocktail solution with said array; and d) determining the binding affinity of each labeled ligand to its said paired receptor.

19. A method for profiling or screening a compound or target, the method comprising: a) providing an plurality of receptor microspots on a solid surface to form an array; b) preparing a cocktail solution of labeled ligands, each labeled ligand having an affinity to bind with at least one corresponding paired receptor in said array; wherein a compound is either present or absent from said cocktail solution; c) contacting said cocktail solution with said array; and d) determining the potency of said compound against the binding of each labeled ligand to its said paired receptor.

20. The method according to claim 19, wherein the method further comprises including multiple compounds in said cocktail solution, wherein each compound binds with a single receptor in said array; and determining the relative potency of each said compound against the binding of each labeled ligand to its said paired receptor.

21. The method according to claim 19, wherein said compound or target is a biological molecule, biochemical or chemical entity, molecule, or pharmaceutical drug candidate to be detected.

22. The method according to claim 19, wherein said biological molecule includes a modified or unmodified nucleotide, nucleoside, peptide, polypeptide, protein, lipid, or saccharides.

23. The method according to claim 19, wherein said method involves providing at least two receptors co-immobilized within a single microspot, in combination with a two-color detection technique, to at least double the capacity of probe elements.

24. The method according to claim 23, wherein said method involves providing four or more receptors co-immobilized within a single microspot, in combination with a four or more color detection technique, to at least double the capacity of probe elements.

25. A method for profiling or screening a compound or target, the method comprising: a) providing an plurality of receptor microspots on a solid surface to form an array; b) preparing a cocktail solution of labeled ligands, each labeled ligand having an affinity to bind with at least one corresponding paired receptor in said array; c) contacting said cocktail solution with said array; d) determining the total binding signal of each receptor; e) sequentially contacting said array with a second solution containing a compound; and f) determining the amount of pre-bound labeled ligands to each receptor displaced by said compound.

26. The method according to claim 25, wherein said method involves providing at least two receptors co-immobilized within a single microspot, in combination with a two-color detection technique, to at least double the capacity of probe elements.

27. The method according to claim 26, wherein said method involves providing four or more receptors co-immobilized within a single microspot, in combination with a four or more color detection technique, to at least double the capacity of probe elements.

28. A thematic multiplexed GPCR microarray, the microarray comprising: a plurality of GPCRs arranged on a substrate at positionally defined locations; said GPCRs are either a) a member from each subfamily of GPCRs, or b) at least a single member selected from several related subfamilies of GPCRs, or c) a GPCR and its mutants or its corresponding GPCRs originated from different species.

29. The microarray according to claim 28, wherein each set of said GPCRs in said microarray are related to each other by either functionality, physiology, family, pathology, tissue-distribution, or mutagenesis.

30. The microarray according to claim 28, wherein said at least two receptors are co-immobilized within a single microspot, in combination with a two-color detection technique, to at least double the capacity of probe elements.

31. The microarray according to claim 30, wherein four or more receptors are co-immobilized within a single microspot, in combination with a four or more color detection technique, to at least double the capacity of probe elements.

32. The microarray according to claim 28, wherein said GPCRs are motilin receptor, delta2 opioid receptor, and neurotensin receptor subtype 1.

33. A method to screen or profile a compound using a multiplexed binding assay, the method comprises: a) providing an plurality of GPCR microspots on a solid surface to form an array; b) preparing a cocktail solution of labeled ligands, each labeled ligand having an affinity to bind with at least one corresponding paired GPCR in said array, in either the absence or presence of a compound; c) contacting said cocktail solution with said array; and d) determining the binding profile of said compound against said labeled ligand to its said paired receptor.

34. The method according to claim 33, wherein said GPCR are motilin receptor, delta2 opioid receptor, and neurotensin receptor subtype 1.

35. The method according to claim 33, wherein said cocktail solution of labeled ligands contains Bodipy-TMR-motilin 1-16, Cy5-neurotensin 2-13, and Cy5-naltrexone.

36. The method according to claim 33, wherein said method involves providing at least two receptors co-immobilized within a single microspot, in combination with a two-color detection technique, to at least double the capacity of probe elements.

37. The method according to claim 36, wherein said method involves providing four or more receptors co-immobilized within a single microspot, in combination with a four or more color detection technique, to at least double the capacity of probe elements.

38. A method to screen or profile a compound using a multiplexed binding assay, the method comprises: a) providing an plurality of receptor microspots on a solid surface to form an array; b) preparing a solution containing at least one compound; c) contacting said array with said solution; d) detecting the binding of said compound to said receptors; and c) determining the binding profile of said compound to its said paired receptor.

39. The method according to claim 38, wherein said detecting step is based on an evanescent-field detector employing a grating-coupled waveguide, surface-plasmon resonance, or other mass-based biosensor system.

40. The method according to claim 38, wherein said compound can be either unlabeled or labeled.

41. The method according to claim 40, wherein when said compound is labeled said label is a species that may have a sufficient mass or an optical charactertistic useful for enhanced detection sensitivity.

42. The method according to claim 41, wherein said label is a gold particle with a diameter of about 1 nanometer to about 100 nanometers.
